(19) United States
(12) Patent Application Publication
(10) Pub. No.: US 2002/0090711 A1
(43) Pub. Date: Jul. 11, 2002

(54) Process for preparing latent antithrombin III

(76) Inventor: Goran Karlsson, Vallingby (SE)

Correspondence Address: STERNE, KESSLER, GOLDSTEIN & FOX PLLC, 1100 NEW YORK AVENUE, N.W., SUITE 600, WASHINGTON, DC, 20005-3934, US

(21) Appl. No.: 09/986,464

(22) Filed: Nov. 8, 2001

Related U.S. Application Data
(60) Provisional application No. 60/252,148, filed on Nov. 20, 2000.

(51) Int. Cl. C12N 9/64

(52) U.S. Cl. 435/226

(57) ABSTRACT

A process for the preparation of latent antithrombin III is provided. The process comprises incubation of a solution of native antithrombin III in the presence of sulfate ions and a buffer selected from the Good's zwitterionic buffers.

### CROSS-REFERENCE TO RELATED APPLICATIONS

This application claims priority under 35 U.S.C. §119 from prior U.S. Provisional application no. 60/252,148, filed Nov. 20, 2000, which is incorporated herein by reference.

### BACKGROUND OF THE INVENTION

1. Field of the Invention

The present invention relates to a process for the preparation of latent antithrombin III.

2. Description of Related Art

Antithrombin III (AT) is a plasma glycoprotein having a total molecular weight of 58.1 kDa (Lebing et al, Vox Sang. 67, 117-124, 1994), that inhibits serine proteases in the coagulation cascade and thus plays a major role in the regulation of blood clotting. Antithrombin III is an inhibitor of Factors IXa, Xa, XI and XIIa, as well as of thrombin. Thus, AT regulates clot formation in different stages of the coagulation cascade. A small decrease in AT content in the blood is associated with an increased risk of thromboembolism. Concentrates of AT are used in the prophylaxis and treatment of thromboembolic disorders in patients with acquired or hereditary antithrombin deficiency. In addition, it has been reported that AT has a function in many other processes of the human body, for example in angiogenesis and in inflammatory responses. The function of AT in these physiological processes is not fully understood.

A particular form of antithrombin III, which was first characterized by Wardell et al (Biochemistry 36, 13133-13142, 1997), is known as the latent form (L-AT). L-AT and a selectively elastase cleaved variant have been shown to possess a strong antiangiogenic activity, and also to suppress tumor growth in mice that have been injected subcutaneously with a human neuroblastoma cell line (O'Reilly et al, Science 285, 1926-1928, 1999, and WO 00/20026). Hence, L-AT must be considered a potential human anticancer drug. However, clinical evaluation of this potential drug remains to be performed.

Purification of AT with affinity chromatography is done using purified heparin as solid phase bound ligand, as is known in the art. Miller-Andersson et al (Thrombosis Research 5, 439-452, 1974) discloses the use of heparin-Sepharose to purify human AT. This chromatographic system has also been useful for the separation between AT and L-AT, where the decreased affinity of heparin for L-AT relative to AT makes it possible to resolve the two components, as described by Chang and Harper (Thrombosis and Haemostasis 77, 323-328, 1997). Hydrophobic interaction chromatography has been used for the separation of native and latent forms of AT (Karlsson, G & Winge, S. (2001) Protein Expr. Purif. 21:149-155)

Induction of the latent form of AT has previously been performed as described by Wardell et al (supra), who obtained 50-60% L-AT by incubating AT in 0.25 M citrate, 10 mM Tris/HCl, pH 7.4, for 15 h in 60° C.

Upon incubation of native antithrombin III at 60° C. in medium or buffer only, aggregates of polymerized protein are often formed. The presence of these aggregates is detrimental to a high yield of latent antithrombin III, and should be avoided as far as possible.

### BRIEF SUMMARY OF THE INVENTION

The aforementioned and other objects of the invention are met by a process as defined in the claims. Thus, a process is provided, which comprises incubation of a solution of native antithrombin III in the presence of sulfate ions and a buffer selected from Good's zwitterionic buffers. It has surprisingly been found that these incubation conditions makes possible the recovery of latent antithrombin III (L-AT) from the process in yields that are substantially higher than those obtained by methods of the prior art (notably the citrate conditions of Wardell et al), while avoiding possible aggregation problems.

The invention offers several advantages. For example, the invention provides a process for obtaining a high yield of latent antithrombin III relative to the yield obtained with a previously-described method(s). Additionally, the invention offers the advantage of minimizing or reducing the production of aggregates of AT polymers relative to a previously-described method(s). Further, the method of the invention advantageously using commonly available reagents and buffer solutions in an in vitro method. Additionally, the method of the invention can readily be scaled up for industrial production of L-AT. Other features and advantages of the invention will be apparent from the detailed description of the invention and from the claims.

All publications, patents, and patent applications cited herein are incorporated herein by reference.

### BRIEF DESCRIPTION OF THE DRAWINGS/FIGURES

FIGS. 1A-1D: Heparin affinity chromatography of antithrombin using a sodium chloride gradient, 0-2 M (5-60 min). The injected amount of protein was 100 μg for sample A-B, and 150 μg for sample C-D. All samples were incubated at 60° C. for 16 h, except for the reference AT sample A, which was not heat-treated (sample 7 in the example). Sample B (sample 6 in the example) was incubated according to Wardell, ie in 0.5 M citrate. Samples C (sample 2 in the example) and D (sample 1 in the example) were incubated in 5 mM HEPES, pH 7.4, with 0.9 and 0.8 M ammonium sulfate respectively. Integration of the low affinity heparin-binding peak, eluting at 22 min, gave 44%, 71% and 89% of the total integrated area for samples B, C, and D, respectively. Native AT eluted at 39 min.

FIG. 2: Native electrophoresis of antithrombin samples, using 12.5% polyacrylamide in a homogeneous gel. The amount of sample was 0.5 μg protein/lane, and the gels were silver-stained after running. All samples, except for lane 7, were incubated in 60° C. for 16 h.

Lane 1) 5 mM HEPES, 0.8 M ammonium sulfate, pH 7.4

Lane 2) 5 mM HEPES, 0.9 M ammonium sulfate, pH 7.4

Lane 3) 5 mM HEPES, 1.1 M ammonium sulfate, pH 7.4

Lane 4) 5 mM HEPES, 1.4 M ammonium sulfate, pH 7.4

Lane 5) 5 mM HEPES, 2.0 M ammonium sulfate, pH 7.4

Lane 6) 10 mmol Tris/HCl, 0.5 M trisodium citrate, pH 7.4 (according to Wardell et al. 1997)

Lane 7) Reference AT sample, not heat-treated

Lane 8) 25 mM sodium phosphate, 100 mM sodium chloride, pH 7.4

Lane 9) 25 mM HEPES, 0.8 M ammonium sulfate, pH 7.4

Lane 10) 5 mM HEPES, 0.5 M ammonium sulfate, pH 7.4

Lane 11) 5 mM HEPES, 2.0 M ammonium sulfate, pH 7.4

Lane 12) 5 mM HEPES, 0.8 M ammonium sulfate, pH 7.0

All lane numbers correspond to the sample numbers listed in the example below.

### DETAILED DESCRIPTION OF THE INVENTION

The invention provides a process for the preparation of latent antithrombin III (referred to as L-AT), starting from a solution of antithrombin III in its native form (referred to as AT). AT can be isolated from blood plasma by heparin-Sepharose chromatography as has been described. Other suitable methods for isolating AT also are known. For example, hydrophobic interaction chromatography can be used to separate native and latent forms of AT (Karlsson and Winge, Protein Expr. Purif. 21:149-155 (2001)). According to the invention, the AT is then incubated in the presence of sulfate ions and a buffer. The incubation temperature and duration can be readily determined by the skilled person, but normal pasteurization conditions, such as a temperature of about 60° C. for about 16 hours, have been found to work well. The volume of the solution is not critical.

The sulfate ions are preferably provided in the form of a sulfate salt. Here, the use of an alkali metal sulfate, an alkaline earth sulfate or ammonium sulfate is preferred. Especially preferred is the use of ammonium sulfate. A suitable concentration of sulfate ions in the process according to the invention lies in the range from 0.5 to 2.0 M, preferably from 0.7 to 1 M, a concentration between 0.8 and 0.9 M being most preferred.

Another component of the incubation mixture is a buffer selected from Good's zwitterionic buffers (Good et al, Biochemistry 5, 467-477, 1966). Which of the indicated buffers to use in the process of the invention can be determined without undue experimentation, keeping in mind that the buffer should fulfill most or all of the following requirements: it should exhibit a pKₐ value of between about 6 and about 9, a maximum solubility in water and a minimum solubility in other solvents, produce a minimum of salt effects, be stable at the experimental conditions used, and not absorb light in the visible or ultraviolet spectral regions (so as not to interfere with spectrophotometric measurements). Good's zwitterionic buffers, including buffers such as HEPES, MES and PIPES, typically present the desired characteristics. The use of HEPES is particularly preferred in the process according to the invention. The widely used Tris buffer is unsuitable for the purposes of the invention. Preferred buffer concentrations are somewhat dependent on the buffer chosen, but typically lie in the range from 1 to 25 mM, more preferably from 2.5 to 10 mM, most preferably from 4 to 6 mM.

As indicated above, the pH of the incubation reaction should lie between pH 6 and pH 9, preferably between pH 7 and pH 8, most preferably between pH 7.4 and pH 7.6.

Following the incubation of AT under the conditions outlined above, separation of the L-AT thus obtained from remaining AT is preferably performed using heparin affinity chromatography. The L-AT exhibits a lower binding affinity to heparin than AT, eluting substantially faster and enabling easy separation of the two forms of antithrombin III. Alternatively, hydrophobic interaction chromatography can be used.

The preparation of L-AT thus obtained is advantageously subjected to treatment for the inactivation or removal of pathogens, particularly in the form of viruses and prions. This can be done in any stage of the process using one of several methods for inactivation or removal known in the art, or combinations of such methods. Examples of such methods include chemical inactivation, heat inactivation, light inactivation, microwave inactivation and nano-filtration removal. A dead-end filtration procedure with a high salt content, like that described in WO96/00237, is particularly preferred, alone or in combination with other procedures. The removal and inactivation of pathogens can also be performed when the antithrombin III molecules are in the native state, before conversion to L-AT.

The invention is further illustrated by the following, non-limiting example.

### EXAMPLES

A laboratory sample of AT, >95% pure, was obtained from Plasma Products, Pharmacia, Stockholm, Sweden. This sample was prepared according to known methods (Miller-Andersson et al, supra) and used for induction of the latent form of antithrombin.

Preparation of L-AT

The laboratory sample of AT was transferred to the following solutions:

Sample 1) 5 mM HEPES, 0.8 M ammonium sulfate, pH 7.4

Sample 2) 5 mM HEPES, 0.9 M ammonium sulfate, pH 7.4

Sample 3) 5 mM HEPES, 1.1 M ammonium sulfate, pH 7.4

Sample 4) 5 mM HEPES, 1.4 M ammonium sulfate, pH 7.4

Sample 5) 5 mM HEPES, 2.0 M ammonium sulfate, pH 7.4

Sample 6) 10 mmol Tris/HCl, 0.5 M trisodium citrate, pH 7.4 (according to Wardell et al. 1997)

Sample 7-8) 25 mM sodium phosphate, 100 mM sodium chloride, pH 7.4

Sample 9) 25 mM HEPES, 0.8 M ammonium sulfate, pH 7.4

Sample 10) 5 mM HEPES, 0.5 M ammonium sulfate, pH 7.4

Sample 11) 5 mM HEPES, 2.0 M ammonium sulfate, pH 7.4

Sample 12) 5 mM HEPES, 0.8 M ammonium sulfate, pH 7.0

Sample 13) 5 mM HEPES, 0.8 M ammonium sulfate, pH 7.8

All buffers listed above were adjusted to the desired pH at room temperature; 1 M HCl was used for adjustment of sample 6, while 1 M sodium hydroxide was used for pH adjustment of all other samples.

AT at a final concentration of 6 mg/ml was incubated in the solutions (samples 1- 13) in glass tubes for 16 h at 60° C. (except for sample 7, which was kept in a fridge at about 8° C.) and transferred to a solution containing 50 mM Tris/HCl, 50 mM sodium chloride, pH 7.4, using small gel filtration columns (NAP-5 Amersham Pharmacia Biotech, Uppsala, Sweden).

The formation of L-AT in the samples was analyzed by heparin affinity chromatography, and the presence of aggregates was analyzed by native electrophoresis.

Heparin Affinity Chromatography

This method was performed based on Chang and Harper (supra). A HPLC equipped with an TSK Heparin® column (Tosohaas, Stuttgart, Germany, 7.5 i.d.×75 mm, 10 μm, 1000 Å) was used. Eluting buffers were 20 mM Tris/HCl buffer, pH 7.4 (buffer A) and 2 M sodium chloride in 20 mM Tris/HCl buffer, pH 7.4 (buffer B). A linear gradient was run (0-5 min of 0% B, 5-60 min 0-100% B, 60-90 min 0% B). The flow rate was 0.4 ml/min and detection was carried out by measuring the absorbance at 280 nm.

Native Polyacrylamide Gel Electrophoresis

Electrophoresis was performed using a 12.5% polyacrylamide homogeneous Phast® gel (Amersham Pharmacia Biotech, Uppsala, Sweden) employing the recommended running parameters. 0.5 μg protein in 1 μl was loaded in each lane. A diamino silver staining was performed according to the booklet from Pharmacia & Upjohn (Phast SysteM™, Technical Note No 2, Two-dimensional electrophoresis with PhastGel™ separation media, Pharmacia LKB Biotechnology AB, Uppsala, Sweden), except that use was made of a slightly stronger fixation solution, containing 50% ethanol, 10% acetic acid and 40% water.

Antithrombin Activity

Sample 2 (incubation in 0.9 M ammonium sulfate) was analyzed regarding biological AT activity with the thrombin chromogenic peptide substrate (S-2238) (Chromogenix, Molndal, Sweden), according to Handeland et al. (Scand J. Haematol. 31, 427-436, 1983). The assay solution consisted of thrombin, heparin, chromogenic substrate and sample, and the response after incubation was recorded as a change in absorbance at 405 nm.

Results

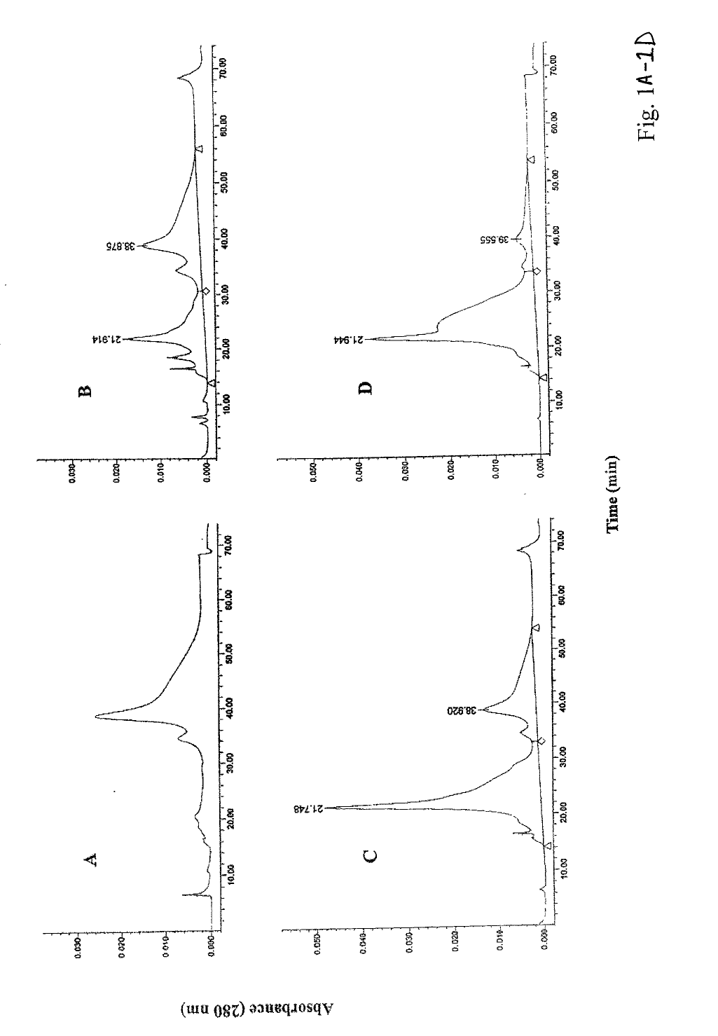

Heparin affinity chromatography gave elution of native AT at 39 min (about 0.9 M sodium chloride) and the main latent peak eluted at 22 min (about 0.3 M sodium chloride) (FIGS. 1A-1B). Integration of the low heparin-binding peak indicated a yield of 44% (FIG. 1B) for the sample prepared according to Wardell's method (sample 6), while incubation in 0.9 and 0.8 M ammonium sulfate (samples 2 and 1, respectively) yielded 71% and 89% respectively of the total integrated area (FIGS. 1C-1D). Table 1 shows that the percentage of formed L-AT decreases at increased concentration of ammonium sulfate/HEPES or at a higher pH value.

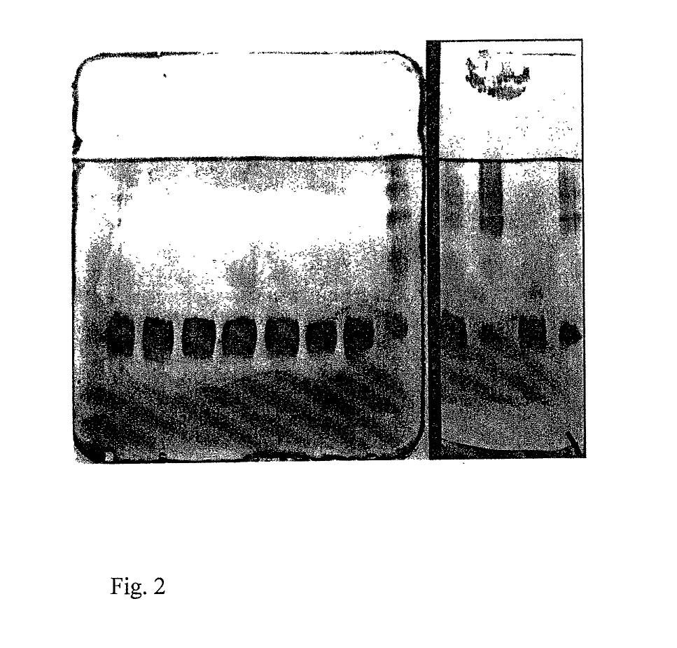

Native electrophoresis of AT incubated at 60° C. in phosphate/NaCl (sample 8) gave a strong formation of aggregates, and only a minor part of the protein remained in the monomeric form (FIG. 2, lane 8). AT incubated according to Wardell (FIG. 2, lane 6), as well as the not incubated AT (FIG. 2, lane 7), gave no aggregates. Incubation in 0.5 M ammonium sulfate (sample 10) induced a strong aggregation (FIG. 2, lane 10), while 0.8 M (sample 1) only gave a minor part of aggregates (FIG. 2, lane 1). Ammonium sulfate at a concentration of 0.9-2.0 M (samples 2-5) resulted in no visible aggregates (FIG. 2, lanes 2-5). At pH 7.0, a lot of aggregates were observed (FIG. 2, lane 12), while a pH of 7.8 gave a smaller amount of aggregates (data not shown).

Antithrombin activity assay on sample 2 (with 0.9 M ammonium sulfate) showed that 34% of the original specific activity remained; this should be compared with the 29% yield of high affinity heparin-binding AT upon analysis of the same sample by affinity chromatography (Table 1).

TABLE 1
Heparin affinity chromatography. Formation of L-AT in
various sample buffers after 16 h incubation in 60° C.

| | | % AT |
|---|---|---|
| | | with low |
| | Sample | heparin |
| Incubation solution | no¹ | affinity |
| 10 mmol Tris/HCl, 0.5 M citrate, pH 7.4 (Wardell) | 6 | 44* |
| 5 mM Hepes, 0.5 M ammonium sulfate, pH 7.4 | 10 | 99 |
| 5 mM Hepes, 0.8 M ammonium sulfate, pH 7.4 | 1 | 89 |
| 5 mM Hepes, 0.9 M ammonium sulfate, pH 7.4 | 2 | 71* |
| 5 mM Hepes, 1.1 M ammonium sulfate, pH 7.4 | 3 | 56* |
| 5 mM Hepes, 1.4 M ammonium sulfate, pH 7.4 | 4 | 49* |
| 5 mM Hepes, 2.0 M ammonium sulfate, pH 7.4 | 5 | 48* |
| 25 mM Hepes, 0.8 M ammonium sulfate, pH 7.4 | 9 | 70 |
| 5 mM Hepes, 0.8 M ammonium sulfate, pH 7.0 | 12 | 99 |
| 5 mM Hepes, 0.8 M ammonium sulfate, pH 7.8 | 13 | 65 |

¹According to the example
*No visible aggregates when analyzed by native electrophoresis (see FIG. 2)

Experimental Conclusions

By incubation of AT in 5 mM HEPES, pH 7.4, containing 0.8 or 0.9 M ammonium sulfate in 60° C. for 16 h, about 85-90% and 70-75% respectively of AT was transformed to the latent form. Native electrophoresis showed a small part of aggregates at 0.8 M ammonium sulfate and no visible aggregates at 0.9 M. In a purification procedure, such small amounts of aggregate can be easily removed by gel filtration or similar techniques.

The optimal concentration for the conversion of AT to L-AT using ammonium sulfate is 0.8-0.9 M. The conversion will also yield good results between 0.7 and 1 M, and some results between 0.5 and 2.0 M. For formation of L-AT, a process using 0.5-2.0 M ammonium sulfate, preferably 0.8-0.9 M, and up to 25 mM HEPES, preferably not more than 10 mM, at a pH near 7.4 has been found to give the most pleasing results. The percentage of L-AT formed will decrease at a higher concentration of ammonium sulfate/HEPES or at a higher pH value. In addition, for the prevention of formation of aggregates, it is necessary not to use too low an ammonium sulfate concentration or too low a pH value. Preferably, the ammonium sulfate concentration is not lower than 0.2 M.

What is claimed is:

1. A process for the preparation of latent antithrombin III, comprising incubating a solution of native antithrombin III in the presence of sulfate ions and a buffer selected from the Good's zwitterionic buffers.

2. A process according to claim 1, wherein said sulfate ions are provided as a salt selected from the group consisting of ammonium sulfate, alkali metal sulfates and alkaline earth sulfates.

3. A process according to claim 2, wherein said sulfate salt is ammonium sulfate.

4. A process according to claim 1, wherein the concentration of said sulfate ions is from 0.5 to 2.0 M.

5. A process according to claim 4, wherein the sulfate ion concentration is from 0.7 to 1 M.

6. A process according to claim 5, wherein the sulfate ion concentration is from 0.8 to 0.9 M.

7. A process according to claim 1, wherein said buffer comprises a HEPES buffer.

8. A process according to claim 1, wherein the concentration of said buffer is from 1 to 25 mM.

9. A process according to claim 8, wherein the buffer concentration is from 2.5 to 10 mM.

10. A process according to claim 9, wherein the buffer concentration is from 4 to 6 mM.

11. A process according to claim 1, wherein the pH value is from 6 to 9.

12. A process according to claim 11, wherein the pH value is from 7 to 8.

13. A process according to claim 12, wherein the pH value is from 7.4 to 7.6.

14. A process according to claim 1, further comprising treating said latent antithrombin III to inactivate or remove pathogens in or from said latent antithrombin III.

15. The process of claim 14, wherein said pathogen is a virus or a prion.

16. A process according to claim 14, wherein said treatment comprises at least one method selected from the group consisting of chemical inactivation, heat inactivation, light inactivation, microwave inactivation and nano-filtration removal.

17. A process according to claim 1, further comprising isolating said latent antithrombin from said solution.

18. A process according to claim 17, wherein said isolating step comprises affinity chromatography or hydrophobic interaction chromatography.

19. A process according to claim 1, wherein said buffer comprises a MES buffer.

20. A process according to claim 1, wherein said buffer comprises a PIPES buffer.
